(19) United States
(12) Patent Application Publication
(10) Pub. No.: US 2004/0198646 A1
(43) Pub. Date: Oct. 7, 2004

(54) Menthol solutions of drugs

(76) Inventors: Moshe Flashner-Barak, Petach Tikva (IL); E. Itzhak Lerner, Petach Tikva (IL); Vered Rosenberger, Jerusalem (IL); Naomi Moldavski, HaNegev (IL)

Correspondence Address: KENYON & KENYON, ONE BROADWAY, NEW YORK, NY, 10004, US

(21) Appl. No.: 10/781,543

(22) Filed: Feb. 17, 2004

Related U.S. Application Data
(60) Provisional application No. 60/449,246, filed on Feb. 20, 2003.

(51) Int. Cl. A61K 38/13; A61K 31/401; A61K 31/366

(52) U.S. Cl. 514/11; 514/423; 514/460; 514/548

(57) ABSTRACT

The present invention relates to compositions comprising solutions of drugs in menthol, especially drugs that are poorly soluble in water, and to methods for making such compositions.

### FIELD OF THE INVENTION

The present invention encompasses compositions comprising solutions or solid solutions of drugs in menthol, especially drugs that are poorly soluble in water, and to methods for making such compositions.

### BACKGROUND OF THE INVENTION

Several clinically important drugs have limited oral bioavailability and high interpatient variability, resulting in difficulty in obtaining optimum treatment regimens for their use. Reasons for such limited oral bioavailability may include poor solubility in water or biological fluids, poor membrane permeability, efficient MDR (multiple drug resistance) pumps, and/or destructive metabolism in the intestine or the liver. Such metabolic destruction may be by the family of cytochrome P450 enzymes that oxidatively destroy many drugs (e.g. CYP3A4) or by glucuronidation enzymes that help the body eliminate the glucuronide derivatives of the drug in the urine or by excretion in the bile to the feces. High interpatient variability is often associated with the genetic variability of metabolic pathways in humans as well as the genetic variation in the expression of the P-glycoprotein MDR pumps.

Drugs with limited oral bioavailability include cyclosporines. Cyclosporines are a very important family of drugs which are used for the avoidance of organ rejection after organ transplant. Cyclosporines, however, suffer from erratic absorption caused by most of the factors mentioned above. See, A. Lindholm, “Factors Influencing the Pharmacokinetics of Cyclosporine in Man,” Therapeutic Drug Monitoring, 13 (6), 465-477 (1991). Cyclosporines are insoluble in water, are expelled from cells of the intestine by P-glycoprotein efflux pumps, and are heavily metabolized both in the intestine and in the liver by cytochrome P-450 enzymes. Ducharme, et al., “Disposition of Intravenous and Oral Cyclosporine after Administration with Grapefruit Juice,” Clinical Pharmacology and Therapeutics, 57(5), 485-491 (1995); and Wu, et al., “Differentiation of Absorption and First-Pass Gut and Hepatic Metabolism in Humans: studies with Cyclosporine,” Clinical Pharmacology and Therapeutics, 58(5), 449-497 (1995). Since the therapeutic window for cyclosporines is not very wide and the toxic effects of overdose are pronounced dosing with this drug type has traditionally been difficult. See e.g., PHYSICIAN'S DESK REFERENCE, pp. 2310-2313 (57th Ed. 2003).

Cyclosporines were originally formulated in oil-based formulations so as to dissolve the drug. Oil and water do not mix very well, thus adding to the variability of the bioavailability of the product. The use of cyclosporines in microemulsion formulations has somewhat improved this situation, however, the efflux pump and oxidative metabolism issues remain essentially as problematical as before. To address the poor bioavailability of cyclosporines and other drugs, Benet and co-workers described dosing the drug either after, with, or mixed with essential oils or essential oil components such as menthol and carvone among others. See, Benet, et al., U.S. Pat. Nos. 5,665,386; 5,716,928; 6,121,234; 6,004,927; and 6,028,054. Benet showed, using in vitro tests, an inhibition of metabolism of cyclosporine and other drugs and a concomitant improvement in bioavailability. The insolubility of the drug in water and the incompatibility of oil based formulations with the aqueous environment of the human gut were still present.

The pharmacokinetics of cyclosporine have been studied using the classical oil-based formulation and the improved microemulsion formulation along with metabolic inhibitors such as ketoconazole. Aklaghi, et al., “Pharmacokinetics of Cyclosporine in Heart Transplant Recipients Receiving Metabolic Inhibitors,” The Journal of Heart and Lung Transplantation, 20 (4), 431-438 (2001). Ketoconazole inhibits CYP3 metabolism as well as P-glycoprotein efflux pumps. The microemulsion formula gave improved bioavailability and somewhat of an improvement in variability when tested without ketoconazole. Treatment with ketoconazole greatly improved the bioavailability of the cyclosporine but not the variability. When pretreating with ketoconazole, the micro emulsion formulation was no better than the oil based formulation. While concurrent treatment with ketoconazole is practiced in certain medical centers to improve cyclosporine bioavailability, in general, the medical community is against giving potent drugs with serious toxic side effects as an adjuvant for another drug when there is no medical need for its administration. Ketoconazole is a potent anti-fungal which is known to exhibit side effects. The need for a safe alternative that will both raise the bioavailability of cyclosporines and lower the inter-patient variability is still present.

The statin drugs, which are used to treat high cholesterol levels, have become some of the most widely used drugs in the world. The family of statin drugs suffers from poor oral bioavailability. This poor oral bioavailability is believed to be caused, to a great extent, by high first pass metabolism. Simvastatin, one of the most widely used drugs in this class, is a prodrug of its active metabolite. However, only about 5% of the dose is available as the active metabolite in the blood due to hepatic first pass metabolism. MARTINDALE: THE COMPLETE DRUG REFERENCE, pp. 969-970 (33^{rd} Ed., 2002). The statins have serious toxic side effects in terms of muscle disorders, rhabdomyolisis being one of the more serious side effects. Id. As with cyclosporines, the inter-patient variability makes it difficult for the doctor to tailor proper dosage for the patient so as to give effective cholesterol reduction without toxic adverse events. Simvastatin has been administered with grapefruit juice or with a capsule of peppermint oil. Peppermint oil, which is known to inhibit CYP3A4, raised bioavailability 60% while the grapefruit juice, which is known to inhibit both CYP3A4 and the P-glycoprotein efflux pump, raised bioavailability 300%. Wacher, et al., “Peppermint Oil Increases the Bioavailability of Felodipine and Simvastatin,” Clinical Pharmacology and Therapeutics, 71(2), P67 Abstract TPII-95.

Paclitaxel is an important antineoplastic agent that is administered by intravenous injection. Paclitaxel suffers from very poor solubility in water. The insolubility hampers i.v. dosing, causing a need for special formulations which may have non-trivial toxicity profiles. PHYSICIAN'S DESK REFERENCE, pp. 1129-1138 (56^{th} Ed. 2002). The insolubility of paclitaxel also hampers use in oral dosing. This problem, however, is minor in comparison to the effects of the P-glycoprotein efflux pump in the intestine. Paclitaxel has been successfully dosed orally by co-administering it with efficient inhibitors of the P-glycoprotein pump such as cyclosporines. See, Malingre, et al., “The Effect of Different Doses of Cyclosporin A on the Systemic Exposure of Orally Administered Paclitaxel,” Anti-Cancer Drugs, 12, 351-358 (2001); Malingre, et al., “A Phase I and Pharmacokinetic Study of Bi-Daily Dosing of Oral Paclitaxel in Combination with Cyclosporin A,” Cancer Chemother Pharmacol., 47, 347-354 (2001); and Broder, et al., U.S. Pat. Nos. 5,968,972; and 6,395,770. Cyclosporines, however, are much too potent a drug type to be used as an adjuvant for the enhanced availability of another drug, even one as important as paclitaxel. Another method of orally dosing paclitaxel is clearly needed.

Many drugs have glucuronidation as their main metabolic pathway of elimination. GOODMAN AND GILMAN'S: THE PHARMACOLOGICAL BASIS OF THERAPEUTICS, p. 13 (9^{th} ed., 1996); and De Wilt, et al., “Glucuronidation in Humans. Pharmacogenetic and Developmental Aspects,” Clinical Pharmacokinetics, 36(6), 439-452 (1999). Recent evidence shows that this pathway may be important in the metabolism of simvastatin along with the mechanisms described above. Prueksaritanont, et al., “Glucuronidation of Statins in Animals and Humans: A Novel Mechanism of Statin Lactonization,” Drug Metabolism and Disposition, 30, 505-512 (2002). The present invention overcomes many of the existing limitations within the prior art by providing novel formulations.

### SUMMARY OF THE INVENTION

One embodiment of the invention encompasses compositions for increasing the oral bioavailability of a drug comprising at least one poorly bioavailable drug dissolved in an effective amount of menthol. The poorly bioavailable drug may be at least one drug with low aqueous solubility, a drug metabolized by cytochrome P450, a drug expelled from cells by the P-glycoprotein pump, or a drug metabolized via glucuronidation. A drug with low aqueous solubility is a drug having a water solubility of less than about 20 mg/per milliliter of water.

Another embodiment of the invention encompasses compositions wherein the poorly bioavailable drugs include, but are not limited to, at least one of cyclosporine, atorvastatin, cerivastatin, fluvastatin, lovastatin, mevastatin, pravastatin, simvastatin, paclitaxel, fenofibrate, itraconazole, bromocriptine, carbamazepine, diazepam, paclitaxel, etoposide, camptothecin, danazole, progesterone, nitrofurantoin, estradiol, estrone, oxfendazole, proquazone, ketoprofen, nifedipine, verapamil, or glyburide. Preferably, the drug includes cyclosporine, atorvastatin, cerivastatin, fluvastatin, lovastatin, mevastatin, pravastatin, simvastatin, or paclitxel. More preferably, the drug is simvastatin.

Yet another embodiment of the invention encompasses methods for improving the bioavailability of a drug comprising dissolving the drug in an effective amount of menthol. Another method of the invention encompasses methods for improving the oral bioavailability of a drug comprising dissolving at least one poorly bioavailable drug in an effective amount of menthol. The method may further comprise administering the composition to a mammal. In one embodiment, the amount of menthol sufficient to increase the drug's bioavailability may be from about 20% to about 99% by weight, preferably, the menthol may be present in an amount of about 60% to about 95% by weight of the composition. Alternatively, the amount of menthol may be sufficient to increase the oral bioavailability of the drug by an increase of about 10% or more in the average area under the blood or plasma concentration versus time curve (AUC) when compared to the average AUC for a non-menthol containing composition of the drug.

Yet another embodiment of the invention encompasses methods for reducing the variability of the bioavailability of a drug comprising dissolving at least one poorly bioavailable drug in an effective amount of menthol. The method may further comprise administering the composition to a mammal. In one embodiment of the method, the amount of menthol may be sufficient to decrease the variability in the drug's bioavailability by about 10% or more of the relative standard deviation (CV %) of the area under the blood or plasma concentration versus time curve (AUC) when compared to the AUC of a non-menthol containing formulation of the drug.

Another embodiment of the invention encompasses methods for increasing the extent of time that a drug provides a therapeutically significant concentration in blood or plasma comprising dissolving at least one poorly bioavailable drug in an effective amount of menthol. In one embodiment, the amount of menthol may be sufficient to extend the time that the drug provides a therapeutically significant concentration in blood or plasma by one hour or more.

### DETAILED DESCRIPTION OF THE INVENTION

The invention comprises formulations of drugs with low bioavailability and menthol. As used herein, the term “poor bioavailability” or “poorly bioavailable” refers to a drug that has an oral bioavailability in its active form, whether it be the drug as dosed or an active metabolite thereof, of less than 30%.

Not to be limited by theory, it is believed that the compositions of the invention operate, in part, by providing a composition where the poorly bioavailable drugs are combined with compounds that aid solubility and/or compounds that compete with the poorly bioavailable drug in the biodegradable pathway which degrades the poorly bioavailable drugs. The delivery of the poorly bioavailable drugs is improved by using materials that are generally recognized as safe and without the use of potent drugs to establish an efficient competition within the biodegradable pathway. Thus, the non-active compound would be metabolized prior to the active drug. In particular, our studies found that formulating poorly bioavailable drugs as a solution or a solid solution in menthol improved delivery as compared to dosing the drug alone, dosing the drug after a menthol dose or a menthol containing dose (e.g. peppermint oil), or dosing the drug along with a dose of menthol. The compositions of the invention allow for the use of lower doses of drugs that provide the same systemic concentrations of drugs as the currently supplied doses that undergo extensive presystemic metabolism and degradation. Also, the compositions of the invention reduce interpatient variability caused by the inherently differing metabolic profiles between subjects.

Menthol, chemically known as (1α,2β,5α)-5-methyl-2-(1-methylethyl)-cyclohexanol, is partially soluble in water. Because menthol has a low melting point, i.e., about 41° C. to 43° C., compositions of menthol and drugs dissolved within the menthol have melting points close to body temperature. This property allows menthol to act as an efficient solvent for many drugs. We have found that menthol is a superior solvent for poorly water soluble drugs as compared to oil based drug formulations, because, in part, the drugs are more available to the aqueous medium of the gastrointestinal tract as compared to oil based formulations. Although, menthol has been known to act as a skin absorption enhancer, it is believed that menthol may also improve gastro-intestinal drug absorption as well.

The invention advantageously uses menthol in close proximity with poorly bioavailable drugs to deter drug biodegradation in a kinetically competitive environment. In other words, menthol may be used to inhibit biological degradation pathways which metabolize the active drug and/or kinetically compete with the drug at the biologically active degradation site. For example, menthol inhibits CYP3A4 metabolism and the P-glycoprotein pump, thus, menthol in close proximity to and in intimate contact with the poorly bioavailable drug greatly enhances the bioavailability of the drug as the drug does not undergo degradation. Also, menthol which has been shown to be metabolized to a glucuronide derivative, can serve as a sacrificial molecule wherein menthol is degraded prior to the drug, thus delaying drug degradation and extending drug bioavailability. In other words, menthol is potentially capable of competing with a drug as a decoy for glucuronidation, thereby leaving less of the drug metabolized and yielding an overall increase in the drug bioavailability.

The present invention encompasses pharmaceutical compositions for improving the bioavailability of a drug comprising at least one drug dissolved in an effective amount of menthol. In particular, the invention encompasses pharmaceutical compositions for improving the bioavailability of a drug comprising at least one poorly bioavailable drug dissolved in an effective amount of menthol. As used herein, the term “improving bioavailability” refers to the increase in concentration of a drug as compared to the concentration of the drug without menthol. In other words, drug bioavailability is proportional to, and is typically measured by, the total area under the curve (AUC) of the concentration of the drug found in blood or plasma versus time when measured in a pharmacokinetic trial in a human or an animal. The AUC may be expressed as AUCt, i.e. the area under the curve to the last measured time point, or AUC_{I}, i.e. the area under the curve extrapolated to infinite time. The improvement in bioavailability is measured by the percent increase in the average AUC of the subjects in the trial when dosing the drug dissolved in menthol as compared to the average AUC of the same subjects obtained by standard dosing of the drug. Alternately, the AUC ratio of the test formulation (AUCf) to the AUC of the reference formulation (AUCr) may be calculated on a per subject basis and then averaged. A percent of the average ratio (AUCf/AUCr) above 100% is then the improvement in bioavailability. Typically, the improvement in the average AUC when dosing the drug dissolved in menthol as compared to the average AUC obtained by standard dosing of the drug is about 5%, and preferably, the improvement is about 10% or more in the bioavailability, which is considered significant.

The present invention further provides a pharmaceutical composition directed to improving the extent of time that a drug provides a therapeutically significant concentration in blood or plasma and/or reducing the drug bioavailability variability, wherein the drug is dissolved in menthol. As used herein, the term “improving the extent of time” refers to the increase in length of time that a drug provides a therapeutically significant concentration in blood or plasma. Preferably, the time a drug provides a therapeutically significant concentration in blood or plasma is extended by about one hour or more. As used herein, the term “drug bioavailability variability” is defined as the relative standard deviation, expressed as CV %, of the drug's AUC over the subjects tested. A highly variable drug is one with a CV % greater than 50%. An improvement of the CV % by 10 percent or more is considered significant. The present invention is particularly directed to a pharmaceutical composition comprising a solid or solid solution of a drug dissolved in an effective amount of menthol. The solid solution may include a compound or polymer that forms a dispersion with the drug.

The poor bioavailability of the drug may be due to several factors. Such factors include, but are not limited to, low aqueous solubility, metabolism by cytochrome P450, expulsion from cells by the P-glycoprotein pump, or metabolism via glucuronidation. Thus, the present invention encompasses compositions for increasing the bioavailability of drugs with low aqueous solubility, drugs metabolized by cytochrome P450, drugs expelled from cells by the P-glycoprotein pump, and/or drugs metabolized via glucuronidation. As used herein, the term “low aqueous solubility” refers to a drug that is considered to be poorly water-soluble, i.e., the drug has a water solubility of less than about 20 mg/per milliliter of water.

Any pharmacologically active substance or drug can be used in the practice of the present invention. Preferred drugs, however, include drugs having poor bioavailability. Examples of drugs having poor bioavailability include, but are not limited to, cyclosporine, statins, paclitaxel, fenofibrate, itraconazole, bromocriptine, carbamazepine, diazepam, paclitaxel, etoposide, camptothecin, danazole, progesterone, nitrofurantoin, estradiol, estrone, oxfendazole, proquazone, ketoprofen, nifedipine, verapamil, or glyburide. Statins include, but are not limited to, atorvastatin, cerivastatin, fluvastatin, lovastatin, mevastatin, pravastatin, or simvastatin. Preferably, the drugs having poor availability include at least one of cyclosporine, statins, or paclitxel. A more preferred statin is simvastatin. Other examples of drugs having poor bioavailability will be readily apparent to one of ordinary skill in the art.

The amount of drug in the composition of the invention should be sufficient to be therapeutically effective for the condition administered. One of ordinary skill in the art can easily determine with little or no experimentation the effective amount of drug. Typically, the drug is present in an amount of about 5% to about 40% by weight of the composition, preferably, the drug is present in an amount of about 10%.

The amount of menthol in the composition of the invention should be sufficient to improve the bioavailability of the poorly bioavailable drug. Typically, the amount of improvement should be at least about 5% of the average AUC as compared to the average AUC of a non-menthol containing formulation and preferably, the improvement is about 15%. One of ordinary skill in the art can easily determine with little or no experimentation the effective amount of menthol. Typically, menthol is present in the composition in a amount of about 20% to about 99% by weight of the composition, and preferably, menthol is present in an amount of about 60% to about 95%. More preferably, menthol is present in the composition in an amount of about 80% to about 90% by weight.

The compositions of the invention may also encompasses other excipients commonly used in drug manufacture including, but not limited to, binders, fillers, disintegrants, lubricants, colorants, carriers, and diluents.

Another embodiment of the invention encompasses methods of improving the bioavailability of a drug comprising dissolving the drug in an effective amount of menthol. In particular, the invention encompasses methods for improving the bioavailability of a drug comprising dissolving at least one drug with low aqueous solubility, drug capable of being metabolized by cytochrome P450, a drug capable of being expelled from cells by the P-glycoprotein pump, or a drug capable of being metabolized via glucuronidation in an effective amount of menthol. Typically, the amount of improvement should be at least about 5% of the average AUC as compared to the average AUC of a non-menthol containing formulation and preferably about 15%, as explained above.

The invention encompasses methods for reducing the variability of the bioavailability of a drug comprising dissolving at least one drug with low aqueous solubility, a drug capable of being metabolized by cytochrome P450, a drug capable of being expelled from cells by the P-glycoprotein pump, or a drug capable of being metabolized via glucuronidation in an effective amount of menthol. As described above, drug variability is defined as the relative standard deviation, expressed as CV %, of the drug's AUC over the subjects tested. A highly variable drug is one with a CV % greater than 50%. Typically, the reduction is about 5% of the relative standard deviation (CV %) of the area under the blood or plasma concentration versus time curve (AUC) when compared to a non-menthol containing formulation average AUC, and preferably, the decrease in CV % is by about 10% or more, which is considered significant.

Another embodiment of the invention encompasses methods for increasing the extent of time that a drug provides a therapeutically significant concentration in blood or plasma comprising dissolving at least one poorly bioavailable drug in an effective amount of menthol. Typically, the extent of the bioavailability of a drug is increased by the administration of a composition comprising at least one drug and menthol, wherein the menthol is present in an amount sufficient to extend the time that the drug provides a therapeutically significant concentration by one hour or more.

The present invention encompasses unit dosage forms of the pharmaceutical composition comprising a unit dosage form of a drug dissolved in an effective amount of menthol. The compositions of the invention may be administered to a mammal. Preferably, the mammal is a human.

One embodiment encompasses the compositions of the invention be prepared into solid solution dosage forms. In particular, the compositions may be formulated into oral solid dosage forms such as capsules, tablets, or gelcaps. In particular, the pharmaceutical compositions can be made into unit dosage forms.

In one embodiment, the solid solution is formed on the surface of at least one pharmaceutical carrier particle. For example, a molten combination of drug and menthol can be applied to the surface of particles of one or more pharmaceutical carriers, and allowed to cool to form the solid solution on the surface of the pharmaceutical carrier or carriers.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

Cyclosporine (20 g) was heated in menthol (80 g) to 56° C. while stirring until the cyclosporine dissolved yielding a clear solution. Microcrystalline cellulose (Avicel pH 102, 100 g) was added to the clear solution which was cooled to room temperature giving a solid solution of cyclosporine in menthol on the microcrystalline cellulose. The solid was milled using a Quadro Comil milling machine, with screens of 6350, 1575 and 813 microns sequentially used to produce a powder ready for filling into capsules.

### Example 2

Simvastatin (20 g) was heated in menthol (200 g) to 60° C. while stirring at 150 rpm in a jacketed reactor. The simvastatin dissolved in the menthol to give a clear solution. The solution was cooled to room temperature to a solid solution of simvastatin in menthol. The solid solution was milled using a Quadro Comil milling machine with a 1640 micron screen. The powder (200 mg) was filled into #0 capsules. The capsules were assayed for simvastatin content by dissolving a capsule in a pH 4 phosphate buffer containing acetonitrile (1:1). The simvastatin content was assayed on a C-18 column by HPLC and found to contain 20 mg of simvastatin per capsule. The release of simvastatin was measured in 450 ml of pH=7 phosphate buffer containing 0.5% sodium lauryl sulfate (SLS) in water at 37° C. and 50 rpm in an USP apparatus II dissolution system. The release was found to be greater than 75% at 30 minutes.

### Example 3

Raloxifene HCl (60 mg, Evista, ELI LILLY®) was dosed to twelve healthy volunteers either alone or with a capsule containing 180 mg menthol in a crossover fashion with a two week washout between sessions. Blood samples were taken at 0, 0.5, 1, 2, 4, 6, 8, 10, 12, 16, 20, 24, 48, 72 and 96 hours and the content of raloxifene assayed. The average Cmax of the raloxifene dosed with menthol was 36% higher than the reference (320 pg/ml vs. 235 pg/ml), while the average area under the curve (AUC) was 8% higher when dosing with menthol (3041 vs. 12090 pg*hr/ml). Raloxifene is a long half-life drug (t_{1/2} for the test was 26 hours and for the reference was 28 hours), while menthol has a short half-life. Without wishing to be bound by theory or mode of action, it is believed that the main effect of menthol is seen in the first hours where it can effectively compete with the drug for glucuronization. An analysis of the AUC over the first six hours shows that the test AUC is 35% higher than the reference, mirroring the Cmax result. Without wishing to be bound by theory or mode of action, it is believed that dosing with menthol can successfully compete with the metabolism of the drug, yielding a better pharmacokinetic profile.

### Example 4

An open-label study with randomized three-way crossover comparative pharmacokinetic study was conducted with 12 healthy fasted volunteers each receiving a single dose of either: Reference-simvastatin (Simvastatin-Teva®, 20 mg) alone; Test 1—simvastatin (Simvastatin-Teva®, 20 mg)+menthol (180 mg capsule); or Test 2—simvastatin/menthol (10% simvastatin dissolved in menthol, 20 mg of simvastatin per capsule). A dose was administered to each subject on three occasions, separated by at least a 1 week wash-out period between each session. All subjects received both the tests and reference drugs in a three-way crossover design.

Each subjects was randomly assigned at the first study period to either of the Test formulations or to the Reference formulation, and was subsequently crossed over at least one week later to either of the alternative treatments. The process was repeated during the third study session, such that each subject was exposed to one of the following treatment schemes: T₁→R→T₂; T₁→T₂→R; R→T₁→T₂; R→T₂→T₁; T₂→R→₁; T₂→T₁→R.

Drug concentration was determined by taking blood samples from all subjects regardless of treatment assignment at the following time points: 0 hour (pre-dosing), 0.5, 1, 1.5, 2, 3, 4, 6, 8, 10 and 12 hours post-initial dosing, for a total of 11 samples per study. Each sample was tested for simvastatin lactone and simvastatin hydroxyacid, the active metabolite, by analysis using a validated LC/MS/MS method.

The AUCt and AUC_{I}, Cmax, Tmax, and half life (t_{1/2}) were calculated for each volunteer both for simvastatin in plasma and for the active metabolite simvastatin hydroxyacid in plasma. Table 1 illustrates the average values for simvastatin in plasma and compares the values of the two test formulations to the average values obtained with the reference formulation.

TABLE 1
Simvastatin Concentration in Plasma

| | | Test | | Test1/ | Test2/ |
|---|---|---|---|---|---|
| | Test 1 | 2 | Reference | Ref. | Ref. |
| Avg. AUCt (ng * h/ml) | 20.8 | 26.9 | 14.9 | 1.4 | 1.81 |
| Avg. AUC₁ ng * h/ml) | 25.9 | 33.0 | 16.3 | 1.59 | 2.02 |
| Avg. Cmax (ng/ml) | 5.8 | 8.0 | 5.6 | 1.04 | 1.43 |
| Avg. Tmax (hours) | 1.58 | 2.46 | 1.04 | | |
| Avg. t_{1/2} (hours) | 3.99 | 3.10 | 2.03 | | |

Table 1 demonstrates that both test formulations showed improved bioavailability over the simvastatin reference with the sample having 20 mg of simvastatin dissolved in 180 mg of menthol giving even better results than the concomitant dosing of a 20 mg simvastatin tablet along with a capsule of 180 mg of menthol. For AUCt the average improvement in the bioavailability of Test 1 (concomitant separate dosing) the improvement was 40% while the improvement for the drug dissolved in menthol was 81%. The corresponding values for the AUC extrapolated to infinity were 59% and 102%, respectively. Consequently, the dissolved product gave larger improvements than concomitant separate dosing.

The ratio of the AUCt of each test formulation to the reference formulation for each volunteer was calculated (each volunteer being his own control) and the average value of the ratio calculated. These results are illustrated in Table 2.

TABLE 2
Ratio Analysis of AUCt for Simvastatin in Plasma

| Subject | Test 1 | Test 2 | Reference | Test 1/Ref. | Test 2/Ref. |
|---|---|---|---|---|---|
| 1 | 20.78 | 21.43 | 27.75 | 0.749 | 0.772 |
| 2 | 29.54 | 39.70 | 38.96 | 0.758 | 1.02 |
| 3 | 23.53 | 17.62 | 6.76 | 3.48 | 2.61 |
| 4 | 26.89 | 75.45 | 33.12 | 0.812 | 2.28 |
| 5 | 37.32 | 15.08 | 6.68 | 5.59 | 2.26 |
| 6 | 12.7 | 10.85 | 4.31 | 2.95 | 2.52 |
| 7 | 8.18 | 6.26 | 5.42 | 1.51 | 1.16 |
| 8 | 13.48 | 26.46 | 13.85 | 0.975 | 1.91 |
| 9 | 31.96 | 66.15 | 13.53 | 2.36 | 4.89 |
| 10 | 22.29 | 23.63 | 15.29 | 1.46 | 1.55 |
| 11 | 14.66 | 9.55 | 7.75 | 1.89 | 1.23 |
| 12 | 8.79 | 10.68 | 5.17 | 1.70 | 2.07 |
| Mean | 20.8 | 26.9 | 14.9 | 2.02 | 2.02 |
| ±SD | 9.4 | 22.5 | 11.9 | 1.42 | 1.09 |
| CV % | 45.3 | 83.7 | 80.1 | 70.5 | 54 |

Table 2 illustrates the ratio analysis of the AUCt values. Both test formulations showed a more than 100% improvement in bioavailability compared to the reference formulation. The two test formulations gave the same larger improvement. The value for Tmax is somewhat delayed for Test 2 compared to the reference and slightly so for Test 1. The values of t_{1/2} are slightly longer, which may indicate competition by menthol for metabolic pathways that determine the t_{1/2} such a glucuronidation and CYP3A4 pathways.

Table 3 collected the average values for simvastatin hydroxyacid, the active metabolite, in plasma and compared the values of the two test formulations to the average values obtained with the reference formulation.

TABLE 3
Simvastatin Hydroxyacid in Plasma

| | | | | Test 1/ | Test 2/ |
|---|---|---|---|---|---|
| | Test 1 | Test 2 | Reference | Ref. | Ref. |
| Avg AUCt (ng * h/ml) | 10.2 | 12.7 | 8.9 | 1.15 | 1.43 |
| Avg Cmax (ng/ml) | 1.32 | 1.71 | 1.18 | 1.12 | 1.45 |
| Avg Tmax (hours) | 5.5 | 5.4 | 5.4 | | |
| Avg t_{1/2} (hours) | 8.8 | 5.7 | 6.5 | | |

Table 3 illustrates the values for the active metabolite of simvastatin. Both test formulations showed improved bioavailability as expressed as average AUCt. Test 1 (concomitant separate dosing) showed a 15% improvement in the average bioavailability of the active moiety when compared to the reference drug product. Test 2 (concomitant dissolved dosing) showed a 45% improvement in the average AUCt and therefore in average bioavailability.

The ratio of AUCt of each test formulation to the reference formulation for each volunteer was calculated (each volunteer being his own control) and the average value of the ratio calculated. These results are illustrated in Table 4.

TABLE 4
Ratio Analysis of AUCt for Simvastatin Hydroxyacid in Plasma

| Subject | Test 1 | Test 2 | Reference | Test 1/Ref. | Test 2/Ref. |
|---|---|---|---|---|---|
| 1 | 9.75 | 7.44 | 10.49 | 0.927 | 0.710 |
| 2 | 17.28 | 19.10 | 25.99 | 0.665 | 0.735 |
| 3 | 9.57 | 11.66 | 5.20 | 1.84 | 2.24 |
| 4 | 7.85 | 18.49 | 8.04 | 0.976 | 2.30 |
| 5 | 13.23 | 9.05 | 5.52 | 2.40 | 1.64 |
| 6 | 7.61 | 11.3 | 5.18 | 1.47 | 2.18 |
| 7 | 9.55 | 10.88 | 7.29 | 1.31 | 1.49 |
| 8 | 3.76 | 5.46 | 4.18 | 0.900 | 1.31 |
| 9 | 9.65 | 17.77 | 4.70 | 2.06 | 3.78 |
| 10 | 19.24 | 21.77 | 16.37 | 1.18 | 1.22 |
| 11 | 12.69 | 13.61 | 10.97 | 1.16 | 1.24 |
| 12 | 2.64 | 6.37 | 2.73 | 0.967 | 2.33 |
| Mean | 10.2 | 12.7 | 8.9 | 1.32 | 1.77 |
| ±SD | 4.9 | 5.4 | 6.6 | 0.53 | 0.85 |
| CV % | 47.5 | 42.5 | 73.9 | 39.8 | 48.1 |

Table 4 illustrates the ratio analysis for the AUCt values for the active moiety. Both test formulations showed a clear improvement in the average of the individual ratios of AUCt with Test 2 being superior to Test 1. Test 1 showed an improved ratio of 32% compared to the reference drug product while Test 2 showed a 77% improvement. The variability of the drug absorption for the active moiety is also clearly improved when dosing with menthol. The reference had a percent coefficient of variation of 74% while Test 1 showed 48% and Test 2 43%, both a considerable improvement and Test 2 being superior.

Therefore, administering simvastatin with menthol can significantly improve the bioavailability of both the parent drug and its active metabolite and delivering the drug when dissolved in the menthol gives an even greater improvement in the improved bioavailability, and a lower variability of the active moiety. The approximate 80 to 100% improvement in the bioavailability of the simvastatin itself and the simvastatin hydroxyacid active moiety along with lowered variability should be able to lead to improved dosing and treatment with this important drug.

What is claimed is:

1. A composition for improving the bioavailability of a drug comprising at least one poorly bioavailable drug dissolved in an effective amount of menthol.

2. The composition according to claim 1, wherein the poorly bioavailable drug is a drug with low aqueous solubility, a drug metabolized by cytochrome P450, a drug expelled from cells by the P-glycoprotein pump, or a drug metabolized via glucuronidation.

3. The composition according to claim 2, wherein the drug with low aqueous solubility is a drug having a water solubility of less than about 20 mg/per milliliter of water.

4. The composition according to claim 1, wherein the drug is cyclosporine, atorvastatin, cerivastatin, fluvastatin, lovastatin, mevastatin, pravastatin, simvastatin, paclitaxel, fenofibrate, itraconazole, bromocriptine, carbamazepine, diazepam, paclitaxel, etoposide, camptothecin, danazole, progesterone, nitrofurantoin, estradiol, estrone, oxfendazole, proquazone, ketoprofen, nifedipine, verapamil, or glyburide.

5. The composition according to claim 1, wherein the drug is cyclosporine, atorvastatin, cerivastatin, fluvastatin, lovastatin, mevastatin, pravastatin, simvastatin, or paclitxel.

6. The composition according to claim 1, wherein the compound is simvastatin, paclitaxel, or cyclosporine.

7. A method for improving the bioavailability of a drug comprising dissolving the drug in an effective amount of menthol.

8. A method for improving the bioavailability of a drug comprising dissolving at least one poorly bioavailable drug in an effective amount of menthol.

9. The method according to claim 8, wherein the poorly bioavailable drug is a drug with low aqueous solubility, a drug capable of being metabolized by cytochrome P450, a drug capable of being expelled from cells by the P-glycoprotein pump, or a drug capable of being metabolized via glucuronidation.

10. The method according to claim 8, further comprising administering the composition to a mammal.

11. The method according to claim 8, wherein the amount of menthol is sufficient to increase the oral bioavailability of the drug by an amount represented by an about 10% or more increase in the average area under the blood or plasma concentration versus time curve (AUC) when compared to a non-menthol containing formulation AUC.

12. The method according to claim 9, wherein the amount of menthol is about 60% to 99% by weight.

13. A method for reducing the variability of the bioavailability of a drug comprising dissolving at least one poorly bioavailable drug in an effective amount of menthol.

14. The method according to claim 13, wherein the poorly bioavailable drug is a drug with low aqueous solubility, a drug capable of being metabolized by cytochrome P450, a drug capable of being expelled from cells by the P-glycoprotein pump, or a drug capable of being metabolized via glucuronidation.

15. The method according to claim 13, further comprising administering the composition to a mammal.

16. The method according to claim 13, wherein the amount of menthol is sufficient to decrease the variability in the drug's bioavailability by about 10% or more of the relative standard deviation (CV %) of the area under the blood or plasma concentration versus time curve (AUC) when compared to a non-menthol containing formulation AUC.

17. A method for increasing the extent of time that a drug provides a therapeutically significant concentration in blood or plasma comprising dissolving at least one poorly bioavailable drug in an effective amount of menthol.

18. The method according to claim 17, wherein the poorly bioavailable drug is a drug with low aqueous solubility, a drug capable of being metabolized by cytochrome P450, a drug capable of being expelled from cells by the P-glycoprotein pump, or a drug capable of being metabolized via glucuronidation.

19. The method according to claim 17 wherein the amount of menthol is sufficient to extend the time that the drug provides a therapeutically significant concentration in blood or plasma by one hour or more.
